(19) United States
(12) Patent Application Publication
(10) Pub. No.: US 2026/0224534 A1
(43) Pub. Date: Aug. 6, 2026

(54) ALPHA-2-ADRENERGIC AGONISTS FOR IMPROVING VISION

(71) Applicant: Bausch + Lomb Ireland Limited, Dublin (IE)

(72) Inventors: Scott M. WHITCUP, Irvine, CA (US); Michael E. GARST, Newport Beach, CA (US)

(21) Appl. No.: 19/154,750

(22) PCT Filed: Feb. 6, 2024
(86) PCT No.: PCT/US2024/014669

§ 371 Date: Aug. 7, 2025

Related U.S. Application Data
(60) Provisional application No. 63/483,843, filed on Feb. 8, 2023.

(51) Int. Cl. A61K 31/4174; A61P 27/06

(52) U.S. Cl. CPC A61K 31/4174; A61P 27/06

(57) ABSTRACT

Disclosed herein is a method of improving visual acuity, visual field, and other measures of visual function, comprising administering an alpha-2 adrenergic agonist to a mammal in need thereof.

### CROSS REFERENCE TO RELATED APPLICATIONS

This application claims priority to U.S. Provisional Application No. 63/483,843, filed Feb. 8, 2023, which is incorporated by reference herein in its entirety.

### BACKGROUND

Alpha-2 adrenergic agonists are a class of compounds that have been used to treat a number of conditions including hypertension, attention deficit disorders, certain pain conditions, and more recently as adjuncts to sedation in anesthesiology. In ophthalmology, alpha-2 agonists have been predominantly used to decrease intraocular pressure (IOP) in patients with glaucoma.

In the eye, alpha-2 adrenergic receptors are expressed not only in the ciliary body, iris, and other anterior segment structures, but also in the retina. In addition to the ability of alpha-2 agonists to lower IOP, these compounds have been studied as neuroprotective agents to decrease vision loss in patients with glaucoma. It is important to note that many of the alpha-2 agonists decrease pupil size (miosis) and this creates a pinhole effect that improves depth of field and can improve near vision and decrease visual aberrations caused by irregularities of the surface of the eye. In fact, a number of these miotic compounds are being studied to treat presbyopia, a condition of decreased ability to focus up close, and as a means to treat visual obscurations following ocular surgery, by decreasing pupil size (Kesler et al.) (Edwards et al.). However, by decreasing pupil size, these treatments also decrease the amount of light entering the eye and can decrease far vision and limit visual field. The FDA warns that miotics, drugs that decrease pupil size, can cause patients to experience temporary dim or dark vision, and states that patients should be advised to exercise caution in night driving and other hazardous activities in poor illumination (FDA Prescribing Information for Vuity® pilocarpine hydrochloride ophthalmic solution 1.25%). Patients with many ophthalmic diseases including age-related macular degeneration and glaucoma have profound difficulty seeing, especially under dimly lit situations. (see references attached). Any therapy that meaningfully decreases pupil size decreases the amount of light entering the eye and can exacerbate this deficit.

Although there are multiple medications approved for lowering IOP in patients with glaucoma, there are no treatments to preserve or improve visual function in patients with the disease. Studies have demonstrated that low luminance visual acuity is an earlier clinical marker of change in central retinal function than standard visual acuity measurements and that macular damage in mild to moderate glaucoma is associated with decreased visual function under low luminance conditions (Blumberg et al. 2019). In addition, visual complaints in patients with glaucoma are more common in the absence of optimal luminance, especially under low luminance (Bierings et al. 2018). These measurements of vision under low luminance are more representative of real-life lighting conditions that people experience on cloudy days, or at sunrise or sunset, and are not measures of vision in the dark or extremely low light conditions, where patients are fully adapted to the dark before measuring the vision.

### SUMMARY

Here we demonstrate compounds with a surprising improvement in vision, including under low lighting conditions, without a meaningful effect on pupil size. This was demonstrated in improved visual acuity under low luminance lighting conditions and on visual field testing under standard conditions.

This disclosure generally relates to use of alpha-2 adrenergic agonists for improving vision, such as improving visual acuity, such as under low-luminance lighting conditions, and/or improving visual field. In this method, an alpha-2 adrenergic agonist is administered to a mammal in need thereof, or alternatively, to a mammal having normal vision.

Some embodiments include a method of improving visual acuity comprising administering an alpha-2 adrenergic agonist to a mammal in need thereof.

Some embodiments include a method of improving visual acuity under low-luminance lighting conditions comprising administering an alpha-2 adrenergic agonist to a mammal in need thereof, or alternatively, to a mammal having normal vision.

Some embodiments include a method of improving visual field comprising administering an alpha-2 adrenergic agonist to a mammal in need thereof, or alternatively, to a mammal having normal vision.

Some embodiments include a method of improving or delaying progressive loss of a measure of visual function under normal lighting or low-luminance lighting conditions comprising administering an alpha-2 adrenergic agonist to a mammal in need thereof.

In some embodiments, the alpha-2 adrenergic agonist is a compound that does not have a significant effect upon pupil size when administered in an amount effective to improve visual acuity or visual field.

Some embodiments include a method of improving visual acuity or visual field without reducing pupil size, comprising administering an alpha-2 adrenergic agonist to a mammal in need thereof, or alternatively, to a mammal having normal vision, wherein an improvement in visual acuity or visual field is experienced by the mammal without a reduction in pupil size.

### DETAILED DESCRIPTION

This disclosure generally relates to use of alpha-2 adrenergic agonists for improving vision, such as improving visual acuity, such as under low-luminance lighting conditions, improving visual field, improving or delaying progressive loss of other measures of visual function under normal lighting or low-luminance lighting conditions, such as contrast sensitivity under normal lighting or low-luminance lighting, color vision under normal lighting or low-luminance lighting, and/or performance of vision related tasks under normal lighting or low-luminance lighting such as navigating a maze or driving. In this method, an alpha-2 adrenergic agonist is administered to a mammal in need thereof. The improved vision may be experienced by the mammal within a short period of time, such as within or at 2 weeks, within or at 4 weeks, within or at 3 months, within or at 6 months, or within or at a year from the beginning of the treatment.

In some embodiments, the method or use includes reducing use of a low vision device, such as a wearable or handheld low vision device, including, for example, a magnifier, biopic telescopic glasses, magnifying glasses, prismatic eyeglasses, computer software, a tablet or a phone device, a camera, etc., by a human patient. Reduced use includes altogether avoiding use of the low vision device, as well as using the device with reduced frequency, or using a device which provides a lower level of correction, e.g., less magnification.

In some embodiments, the improvement in vision, such as improved visual acuity, such as under low-luminance lighting conditions, and/or improved visual field, is in the absence of a pupillary effect due to constriction of the pupil. In some embodiments, the improvement in vision, such as improved visual acuity, such as under low-luminance lighting conditions, and/or improved visual field, occurs in a human being without a reduction in pupil size, or without a clinically meaningful reduction in pupil size, or with an average change in pupil size that is from about −0.5 mm to about 0 mm, or about −0.2 mm to about 0 mm.

In some embodiments, the improvement in vision, such as improving or delaying progressive loss of other measures of visual function under normal lighting or low-luminance lighting conditions, such as contrast sensitivity under normal lighting or low-luminance lighting, color vision under normal lighting or low-luminance lighting, and/or performance of vision related tasks under normal lighting or low-luminance lighting, such as navigating a maze or driving, is in the absence of a pupillary effect due to constriction of the pupil. In some embodiments, the improvement in vision occurs in a human being without a reduction in pupil size, or with an average change in pupil size that is from about −0.5 mm to about 0 mm, or about −0.2 mm to about 0 mm.

Decreased visual acuity may be determined by a decrease in Early Treatment of Diabetic Retinopathy Study (ETDRS) visual acuity, or by having an ETDRS visual acuity that is below normal.

Patients with improved visual acuity after treatment may have an improvement of ≥5 letters, ≥10 letters, ≥15 letters, or more, in ETDRS visual acuity, as compared to the patient's ETDRS visual acuity prior to treatment. A ≥15 letter increase in ETDRS visual acuity is equivalent a 3-line increase on a standard vision chart.

The patient may be a non-human mammal or a human being. In some embodiments, the mammal is a human being. In some embodiments, the patient is a human being. In some embodiments, the patient is a non-human mammal, such as a dog, a cat, a mouse, a rat, a rabbit, a monkey, a horse, a pig, etc.

In some embodiments, a human patient has an age of 0-18 years, 18-30 years, 30-50 years, 50-65 years, or 65-100 years. In some embodiments, a human patient is female. In some embodiments, a human patient is male.

The mammal, such as a human being, may be experiencing a retinal condition, glaucoma, or another ocular disorder, such as an ophthalmic condition associated with decreased visual acuity. A retinal condition is associated with decreased visual acuity if patients having the retinal condition may also have decreased visual acuity. In some embodiments, the decreased visual acuity may be caused by the retinal condition or ocular disorder. In some embodiments, the mammal, such as a human being, may be experiencing both a retinal condition or ocular disorder, and decreased visual acuity, and the decreased visual acuity is unrelated to the retinal condition or ocular disorder.

Low-luminance visual acuity refers to visual acuity determined using the standard Early Treatment Diabetic Retinopathy Study (ETDRS) chart with a 2.0 log unit neutral density filter placed in front of the eye.

The mammal, such as a human being, may be experiencing a retinal condition or other ocular disorder, such as a retinal condition associated with decreased low-luminance visual acuity. A retinal condition is associated with decreased low-luminance visual acuity if patients having the retinal condition may also have decreased low-luminance visual acuity. In some embodiments, the decreased low-luminance visual acuity may be caused by the retinal condition or ocular disorder. In some embodiments, the mammal, such as a human being, may be experiencing both a retinal condition or ocular disorder, and decreased low-luminance visual acuity, and the decreased low-luminance visual acuity is unrelated to the retinal condition or ocular disorder.

The mammal, such as a human being, may be experiencing a retinal condition or other ocular disorder, such as a retinal condition associated with decreased visual field. A retinal condition is associated with decreased visual field if patients having the retinal condition may also have decreased visual field. In some embodiments, the decreased visual field may be caused by the retinal condition or ocular disorder. In some embodiments, the mammal, such as a human being, may be experiencing both a retinal condition or ocular disorder, and decreased visual field, and the decreased visual field is unrelated to the retinal condition or ocular disorder.

In some embodiments, the mammal, such as the human being, has glaucoma. In some embodiments, the mammal, such as the human being, has ocular hypertension. In some embodiments, the mammal, such as the human being, has age-related macular degeneration. In some embodiments, the mammal, such as the human being, has a retinal condition or ocular condition associated with decreased visual acuity that is not glaucoma, ocular hypertension, or age-related macular degeneration.

In some embodiments, the mammal, such as the human being, has a retinal condition or ocular condition associated with decreased visual acuity, and the retinal condition or ocular condition associated with decreased visual acuity is not glaucoma, ocular hypertension, or age-related macular degeneration.

In some embodiments, the mammal, such as the human being, has a retinal condition or ocular condition associated with decreased low-luminance visual acuity, and the retinal condition or ocular condition associated with decreased low-luminance visual acuity is not glaucoma, ocular hypertension, or age-related macular degeneration.

In some embodiments, the mammal, such as the human being, has a retinal condition or ocular condition associated with decreased visual field, and the retinal condition or ocular condition associated with decreased visual field is not glaucoma, ocular hypertension, or age-related macular degeneration, such as diabetic retinopathy; other forms of macular degeneration, such as geographic atrophy; a retinal degeneration, including inherited retinal diseases like retinitis pigmentosa, radiation retinopathy; retinal disease caused by toxic medications, light or other insults; retinopathy of prematurity; an ischemic retinal disease, including central retinal vein occlusions, branch retinal vein occlusions, central retinal artery occlusion; sickle cell retinopathy; myopia and myopia related retinal degeneration; uveitis or other inflammatory retinal diseases; decreased vision associated with ocular surgery, decreased vision associated with Alzheimer's disease or other neurologic diseases including stroke or cerebral ischemia, decreased visual acuity from ocular surgery; etc.

In some embodiments, in addition to (or as an alternate to) improving visual acuity or visual field, the alpha-2 adrenergic agonist may be effective in treating the retinal condition or ocular condition. In some embodiments, the alpha-2 adrenergic agonist may be effective in treating glaucoma. In some embodiments, the alpha-2 adrenergic agonist may be effective in treating ocular hypertension. In some embodiments, the alpha-2 adrenergic agonist may be effective in treating age-related macular degeneration. In some embodiments, the alpha-2 adrenergic agonist may be effective in treating a retinal condition or ocular condition associated with decreased visual acuity that is not glaucoma, ocular hypertension, or age-related macular degeneration, such as diabetic retinopathy; other forms of macular degeneration, such as geographic atrophy; a retinal degeneration, including inherited retinal diseases like retinitis pigmentosa, radiation retinopathy; retinal disease caused by toxic medications, light or other insults; retinopathy of prematurity; an ischemic retinal disease, including central retinal vein occlusions, branch retinal vein occlusions, central retinal artery occlusion; sickle cell retinopathy; myopia and myopia related retinal degeneration; uveitis or other inflammatory retinal diseases; decreased vision associated with ocular surgery, decreased vision associated with Alzheimer's disease or other neurologic diseases including stroke or cerebral ischemia, decreased visual acuity from ocular surgery; etc.

In some embodiments, the alpha-2 adrenergic agonist may be effective in delaying progression of myopia in the absence of pupil changes.

Alternatively, the alpha-2 adrenergic agonist may be administered to a mammal, such as a human being, with normal vision to provide vision, such as visual acuity, that is better than normal, or better than the mammal's normal vision prior to the treatment.

The terms “treating” or “treatment” broadly includes any kind of treatment activity, including the diagnosis, cure, mitigation, or prevention of disease in man or other animals, or any activity that otherwise affects the structure or any function of the body of man or other animals.

Any suitable alpha-2-adrenergic agonist may be used, such as:

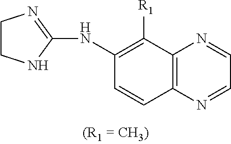

5-methyl-N-(4,5-dihydro-1H-imidazol-2-yl) quinoxaline-6-amine;

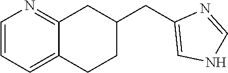 [SMILES: c1cnc2c(c1)CCC(Cc1c[nH]cn1)C2]

7-(imidazole-4-ylmethyl)-5,6,7,8-tetrahydroquinoline;

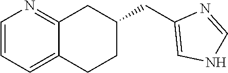 [SMILES: c1cnc2c(c1)CC[C@@H](Cc1c[nH]cn1)C2]

(S)-(+)-7-(imidazole-4-ylmethyl)-5,6,7,8-tetrahydroquinoline;

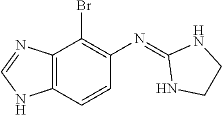 [SMILES: Brc1c(N=C2NCCN2)ccc2[nH]cnc12]

N-(4-bromo-1H-benzo[d]imidazol-5-yl)imidazolidin-2-imine;

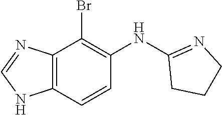 [SMILES: Brc1c(NC2=NCCC2)ccc2[nH]cnc12]

4-bromo-N-(3,4-dihydro-2H-pyrrol-5-yl)-1H-benzo[d]imidazol-5-amine;

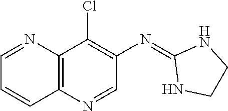 [SMILES: Clc1c(N=C2NCCN2)cnc2cccnc12]

N-(4-chloro-1,5-naphthyridin-3-yl)imidazolidin-2-imine;

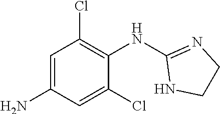 [SMILES: Nc1cc(Cl)c(NC2=NCCN2)c(Cl)c1]

2-(4-amino-2,6-dichloroanilino)-2-imidazoline;

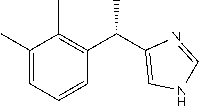

(S)-4-[1-(2,3-dimethylphenyl)ethyl]-3H-imidazole;

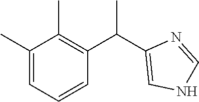

(R,S)-4-[1-(2,3-dimethylphenyl)ethyl]-3H-imidazole;

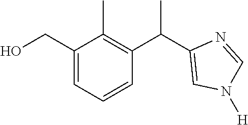 [SMILES: Cc1c(CO)cccc1C(C)c1c[nH]cn1]

(R,S)-(3-(1H-imidazol-4-yl)ethyl-2-methylphenyl)methanol and esters thereof;

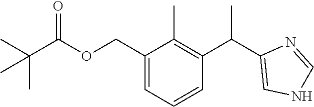 [SMILES: Cc1c(COC(=O)C(C)(C)C)cccc1C(C)c1c[nH]cn1]

[3-(1-(1H-imidazole-5-yl)ethyl)-2-methylphenyl]methyl-2,2-dimethylpropanoate;

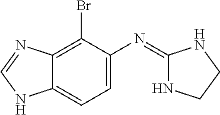 [SMILES: Brc1c(N=C2NCCN2)ccc2[nH]cnc12]

4-bromo-5-(2-imidazolin-2-ylamino)benzimidazole; and

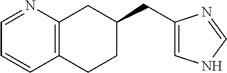 [SMILES: c1cnc2c(c1)CC[C@H](Cc1c[nH]cn1)C2]

(R)-7-(imidazole-4-ylmethyl)-5,6,7,8-tetrahydroquinoline

In some embodiments, the alpha-2 adrenergic agonist is (S)-(3-(1H-imidazol-4-yl)ethyl-2-methylphenyl)methanol, or a pharmaceutically acceptable salt or an ester thereof.

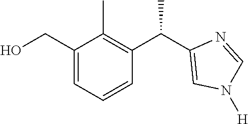 [SMILES: Cc1c(CO)cccc1[C@H](C)c1c[nH]cn1]

### (S)-(3-(1-(1H-imidazol-4-yl)ethyl)-2-methylphenyl)methanol

In some embodiments, the alpha-2 adrenergic agonist is [3-[(1S)-1-(1H-imidazole-5-yl)ethyl]-2-methylphenyl]methyl-2,2-dimethylpropanoate, which is an ester (and may also be a prodrug) of (S)-(3-(1-(1H-imidazol-4-yl)ethyl)-2-methylphenyl)methanol.

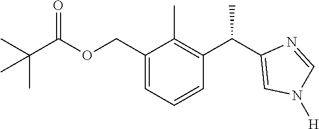 [SMILES: Cc1c(COC(=O)C(C)(C)C)cccc1[C@H](C)c1c[nH]cn1]

### [3-[(1S)-1-(1H-imidazole-5-yl)ethyl]-2-methylphenyl]methyl-2,2-dimethylpropanoate (Compound 1 in Example 1)

Unless otherwise indicated, any reference to a compound herein, such as alpha-2 adrenergic agonist, by structure, name, or any other means, includes pharmaceutically acceptable salts; alternate solid forms, such as polymorphs, solvates, hydrates, etc.; tautomers; deuterium modified compounds, such as deuterium modified dextromethorphan; or any chemical species that may rapidly convert to a compound described herein under conditions in which the compounds are used as described herein.

A pharmaceutically acceptable salt includes a salt that is acceptable for administration to an animal or a human being. Examples of suitable pharmaceutically acceptable salts of the alpha-2 adrenergic agonist include salts of an inorganic acid, e.g., hydrochloric acid, hydrobromic acid, sulfuric acid, phosphoric acid, nitric acid, etc.; or an organic acid such as citric acid, acetic acid, oxalic acid, tartaric acid, succinic acid, malic acid, fumaric acid, ascorbic acid, benzoic acid, tannic acid, palmitic acid, alginic acid, polyglutamic acid, naphthalene-sulfonic acid, napthalenedisulfonic acid, polygalacturonic acid, etc.

The alpha-2 adrenergic agonist may be administered by any suitable route of administration, such as by topical ophthalmic administration (e.g., in the form of eye drops), an injection or implant, such as injections of solutions, suspensions, gels, a sustained-release formulation, an implant that is biodegradable or nonbiodegradable. The injection may be into any suitable location, including an intraocular injection, a subconjunctival injection, a subretinal injection, a suprachoroidal injection, an intracameral injection, a sub-tenons injection, etc. Administration may also be by a systemic route, such as oral or intravenous administration.

The alpha-2 adrenergic agonist may be administered alone, as the sole active pharmaceutical agent being used in a treatment, or it may be administered in combination with one or more other active pharmaceutical agents. Similarly, a dosage form or pharmaceutical composition, such as an eye drop or a sustained release implant, may contain the alpha-2 adrenergic agonist as the sole active pharmaceutical agent in the dosage form or pharmaceutical composition, or the dosage form or pharmaceutical composition may contain the alpha-2 adrenergic agonist and one or more additional active pharmaceutical agents.

In some embodiments, an eye drop containing the alpha-2 adrenergic agonist may be administered to the affected eye of a mammal, such as a human being. For eye drops, another topical ophthalmic route, or oral administration, the alpha-2-adrenergic, such as [3-[(1S)-1-(1H-imidazole-5-yl)ethyl]-2-methylphenyl]methyl-2,2-dimethylpropanoate, (S)-(3-(1-(1H-imidazol-4-yl)ethyl)-2-methylphenyl)methanol, or another compound described herein, may be administered once daily, twice daily, thrice daily, or more frequently. The administration may continue (once, twice, thrice, or more) daily for at least a day, at least 7 days, at least 2 weeks, at least 4 weeks, at least 2 months, at least 3 months, at least 6 months, at least 12 months, at least 2 years, at least 5 years, at least 10 years, about 1-4 weeks, about 1-6 months, about 6-12 months, about 1-3 years, about 3-5 years, up to 10 years, up to 20 years, up to 40 years, up to 80 years, up to 100 years, or longer.

In some embodiments, injected dosage forms or implants, the alpha-2-adrenergic, such as [3-[(1S)-1-(1H-imidazole-5-yl)ethyl]-2-methylphenyl]methyl-2,2-dimethylpropanoate, (S)-(3-(1-(1H-imidazol-4-yl)ethyl)-2-methylphenyl)methanol, or another compound described herein, may be administered once per 1-4 years, 1-3 times per year, 3-6 times per year, 6-12 times per year, etc.

In some embodiments, the alpha-2 adrenergic agonist, such as [3-[(1S)-1-(1H-imidazole-5-yl)ethyl]-2-methylphenyl]methyl-2,2-dimethylpropanoate, (S)-(3-(1-(1H-imidazol-4-yl)ethyl)-2-methylphenyl)methanol, or another compound described herein, is administered in a topical ophthalmic liquid containing about 0.01-1% (w/v), about 0.01-0.04% (w/v), about 0.04-0.06% (w/v), about 0.06-0.1% (w/v), about 0.1-0.2% (w/v), about, about 0.2-0.5% (w/v), or about 0.5-1% (w/v) of the alpha-2 adrenergic agonist.

For topical ophthalmic administration, such as in eye drops, the composition may include ingredients or excipients such buffers, tonicity agents, preservatives, co-solvents, viscosity building agents, etc., in an aqueous or water solution (e.g. deionized water).

Suitable buffers include, e.g., phosphate, bicarbonate, citrate, borate, etc. A topical ophthalmic composition may have any suitable pH, such as about 5-9, about 6-8, about 6.5-7, or about 7-7.5.

Suitable tonicity agents may include, e.g., a salt, such as sodium chloride, potassium chloride, etc.; a sugar, such as glucose, dextrose, glycerin, etc.

Preservatives may help to prevent microbial contamination during use. Suitable preservatives include: stabilized oxychloro complex (sold under the trademark Purite™), stabilized chlorine dioxide, benzalkonium chloride, thimerosal, chlorobutanol, methyl paraben, propyl paraben, phenylethyl alcohol, edetate disodium, sorbic acid, etc. Suitable concentrations of the preservative may be about 0.001-1%, or about 0.01-0.5%, by weight.

The solubility of the components of the present compositions may be enhanced by a surfactant or other appropriate co-solvent in the composition. Such surfactants or cosolvents include polysorbate 20, 60, and 80, Pluronic® F-68, F-84 and P-103, cyclodextrin, Solutol, or other agents known to those skilled in the art. In some embodiments, the surfactant or co-solvent is present at an amount of about 0.01% to 2% by weight.

Viscosity increased above that of simple aqueous solutions may be desirable to increase ocular absorption of the active compound, to decrease variability in dispensing the formulation, to decrease physical separation of components of a suspension or emulsion of the formulation and/or to otherwise improve the ophthalmic formulation. Such viscosity building agents include as examples polyvinyl alcohol, polyvinyl pyrrolidone, methyl cellulose, hydroxypropyl methylcellulose, hydroxyethyl cellulose, carboxymethyl cellulose, hydroxypropyl cellulose or other agents known to those skilled in the art. In some embodiments, the viscosity building agent is present at an amount of about 0.01% to 2% by weight.

Table 1 depicts a suitable eye drop formulation.

TABLE 1

| Component | Amount (% w/v) |
|---|---|
| alpha-2 adrenergic agonist | 0.01-0.5 |
| buffer | 0.1-2 |
| tonicity agent | 0.1-2 |
| Preservative | 0.01-0.5 |
| pH | 7.4 |
| Deionized water | Qs 100 |

The alpha-2 adrenergic agonist may be implanted or injected in a sustained release implant, such as a biodegradable or bioerodible implant, including an implant made of polymeric materials, such as polymers derived from and/or including organic esters and organic ethers, which when degraded result in physiologically acceptable degradation products, including the monomers; polymeric materials derived from and/or including, anhydrides, amides, orthoesters and the like, by themselves or in combination with other monomers; addition or condensation polymers; polymers of hydroxyaliphatic carboxylic acids, either homopolymers or copolymers, and polysaccharides, such as polymers of D-lactic acid, L-lactic acid, racemic lactic acid, glycolic acid, polycaprolactone, and combinations thereof. The alpha-2 adrenergic agonist may be 0-20%, 20-40%, 40-60%, 60-80%, or greater than 80% of the weight of the implant.

In some embodiments, the implant is a polyglycolic acid (PLGA) copolymer having about 0-100% polylactic acid, 15-85% polylactic acid, or about 35-65% polylactic acid. In some implants, a copolymer having about 50% Polylactic acid is used.

### Example 1

Whitecap Biosciences has recently completed a Phase 1/2 study evaluating the safety and IOP-lowering effects of Compound 1 ophthalmic solution in patients with primary open-angle glaucoma or ocular hypertension. Part 1 was an open-label dose escalation study. The purpose of Part 1 of this study (WB007-001), was to assess the safety, tolerability, and IOP-lowering effects of a single drop of an ophthalmic solution containing [3-[(1S)-1-(1H-imidazole-5-yl)ethyl]-2-methylphenyl]methyl-2,2-dimethylpropanoate (Compound 1) with concentrations ranging from 0.05% to 0.4%. Part 2 of the study, was a double-masked, randomized, multi-center active controlled, parallel study assessing the safety and ocular hypotensive efficacy of an ophthalmic solution containing 0.15% Compound 1 and 0.4% Compound 1 compared to 0.5% timolol ophthalmic solution in patients with glaucoma or ocular hypertension. In addition to assessing the efficacy of Compound 1 on lowering IOP, the study also assessed the effects of the drug on visual function.

### Intraocular Pressure

In Part 1 of the study, following a single dose to the study eye, mean change from baseline in IOP (mean±SD) on Day 1 ranged from −0.9±0.9 to −3.4±2.2 mm Hg for the 0.05% Compound 1 group, from −2.1±1.9 to −8.3±4.1 mm Hg for the 0.15% Compound 1 group and from −1.9±2.4 to −8.2±1.9 mm Hg for the 0.4% Compound 1 group. Peak efficacy occurred within 30 minutes to 4 hours across the treatment groups.

The study part 2 met the primary endpoint of statistically significant within group IOP lowering on Day 14, Hour 2 when dosed twice daily. The mean change from baseline analyses in the study eye demonstrated statistically significant IOP-lowering effects in the two Compound 1 treatment groups at all postbaseline timepoints (p≤0.007) and for all postbaseline timepoints for Timolol (p≤0.004). For the primary endpoint (Day 14, Hour 2), the time for the peak effect of Timolol, the mean change from baseline in IOP was −4.9±1.8 mm Hg for subjects receiving 0.15% Compound 1 (p<0,001), −5.1±2.5 mm Hg for subjects receiving 0.4% Compound 1 (p<0.001), and −6.0±3.0 mm Hg (p<0.001) for subjects receiving Timolol.

Additional analyses examining the effect of Compound 1 on visual function included low-luminance visual acuity and visual fields. Dose related improvements in low luminance visual acuity were observed in patients receiving Compound 1. The percentage of subjects achieving an increase in Early Treatment of Diabetic Retinopathy Study (ETDRS) visual acuity of 15 or more letters measured under low luminance in the study eye on at least one study visit was 8.7% in subjects receiving 0.15% Compound 1, 16.7% in subjects receiving 0.4% Compound 1, and in 0% of subjects receiving timolol 0.5%. Visual function improvements in the treated non-study eye corroborated findings from the study eye. The percentage of subjects achieving an increase in ETDRS visual acuity of 15 or more letters measured under low luminance in the non-study eye on at least one study visit was 17.4% in subjects receiving 0.15% Compound 1, 25.0% in subjects receiving 0.4% Compound 1, and in 8.3% of subjects receiving timolol 0.5%.

Improved visual field results with patients receiving Compound 1 compared to patients receiving timolol were also demonstrated in both the study eyes and the treated non-study eyes. Visual field mean deviation (dB) change from Baseline at Day 14 in study eyes (where a positive change represents an improvement of visual fields) was 0.2±1.2 for subjects receiving 0.15% Compound 1, −0.5±2.3 for subjects receiving 0.4% Compound 1, and −1.2±2.3 for subjects receiving Timolol. For the non-study eye, the change in mean deviation (dB) was 0.4±1.5 for subjects receiving 0.15% Compound 1, −0.2±1.4 for subjects receiving 0.4% Compound 1, and −2.0±3.4 for subjects receiving Timolol. The percentage of patients with an improvement of 1 dB or greater is shown in the Table 1.

| | Improvement ≥1 dB | Improvement ≥1 dB in |
|---|---|---|
| Treatment | in Study Eye | Treated Non-Study Eye |
| 0.15% Compound 1 | 9 patients (39%) | 7 patients (30%) |
| 0.4% Compound 1 | 6 patients (25%) | 6 patients (25%) |
| Timolol 0.5% | 2 patients (17) | 0 patients (0%) |

### Pupil Size

The effect of Compound 1 on pupil size was assessed at the baseline visit and on Day 14. Pupil size results from Hour 0 to Hour 2 showed no change in the majority of subjects. There was no meaningful decrease in pupil size for either dose of Compound 1. In study eyes, the mean change in pupil size from Baseline to the end of the study at Day 14 at Hour 0 (the timepoint where vision function was assessed) was 0.0 mm for subjects receiving 0.15% Compound 1, −0.1 mm for subjects receiving 0.4% Compound 1, and −0.1 mm for subjects receiving Timolol. In the treated non-study eyes, the mean change in pupil size from Baseline to the end of the study at Day 14 at Hour 0 (the timepoint where vision function was assessed) was 0.0 mm for subjects receiving 0.15% Compound 1, −0.1 mm for subjects receiving 0.4% Compound 1, and −0.1 mm for subjects receiving Timolol.

### Safety

Both doses of Compound 1 were safe and well-tolerated. The majority of subjects reported the study medications to be “comfortable” or “very comfortable.”

1. A method of improving visual acuity comprising administering an alpha-2 adrenergic agonist to a mammal in need thereof.

2. A method of improving visual acuity under low-luminance lighting conditions comprising administering an alpha-2 adrenergic agonist to a mammal in need thereof.

3. A method of improving visual field comprising administering an alpha-2 adrenergic agonist to a mammal in need thereof.

4. The method of claim 1, 2, or 3, wherein the alpha-2 adrenergic agonist is a compound that does not have a clinically relevant effect upon pupil size when administered in an amount effective to improve visual acuity or visual field.

5. A method of improving or delaying progressive loss of a measure of visual function under normal lighting or low-luminance lighting conditions without reducing pupil size, comprising administering an alpha-2 adrenergic agonist to a mammal in need thereof, wherein an improvement or delay of progressive loss of a measure of visual function under normal lighting or low-luminance lighting conditions is experienced by the mammal without a reduction in pupil size.

6. The method of claim 5, wherein the measure of visual function is contrast sensitivity.

7. The method of claim 5, wherein the measure of visual function is color vision.

8. The method of claim 5, wherein the measure of visual function is performance of vision related tasks.

9. A method of improving visual acuity or visual field without reducing pupil size, comprising administering an alpha-2 adrenergic agonist to a mammal, wherein an improvement in visual acuity or visual field is experienced by the mammal without a clinically meaningful reduction in pupil size.

10. The method of claim 9, wherein the mammal has normal vision.

11. The method of claim 9, wherein the human being has myopia.

12. The method of any one of claims 1-10, wherein the alpha-2 adrenergic agonist is (S)-(3-(1-(1H-imidazol-4-yl)ethyl)-2-methylphenyl)methanol, or a pharmaceutically acceptable salt or an ester thereof.

13. The method of claim 11, wherein the alpha-2 adrenergic agonist is [3-[(1S)-1-(1H-imidazole-5-yl)ethyl]-2-methylphenyl]methyl-2,2-dimethylpropanoate.

14. The method of any one of claims 1-13, wherein the mammal is a human being.

15. The method of claim 14, wherein the human being has glaucoma.

16. The method of claim 14, wherein the human being has ocular hypertension.

17. The method of claim 14, wherein the human being has age-related macular degeneration.

18. The method of claim 14, wherein the human being has a retinal condition associated with decreased low-luminance visual acuity.

19. The method of claim 14, wherein the human being has geographic atrophy.

20. The method of any preceding claim, wherein the improvement in visual function, visual acuity, or visual field is experienced within two weeks of receiving the first dose of the alpha-2 adrenergic agonist.
